(19) United States
(12) Patent Application Publication
(10) Pub. No.: US 2020/0388364 A1
(43) Pub. Date: Dec. 10, 2020

(54) Sentiment Detection Using Medical Clues

(71) Applicant: International Business Machines Corporation, Armonk, NY (US)

(72) Inventors: Nan Liu, San Jose, CA (US); Xianying Liu, Fremont, CA (US); Tongkai Shao, San Jose, CA (US); Rashmi Gangadharaiah, San Jose, CA (US); Feng Wang, Santa Clara, CA (US); Sheng Hua Bao, San Jose, CA (US)

(21) Appl. No.: 16/434,521

(22) Filed: Jun. 7, 2019

(51) Int. Cl. G16H 20/10; G16H 10/60

(52) U.S. Cl. CPC G16H 20/10; G16H 10/60

(57) ABSTRACT

Mechanisms are provided to implement a sentiment analysis mechanism for performing sentiment analysis of a medical event and a drug name within a medical document based on a medical context. The sentiment analysis mechanism analyzes a medical document to identify an occurrence of a medical event associated with a drug name and analyzes contextual content associated with the occurrence of the medical event and the drug name to identify one or more sentiment terms present in the contextual content. The sentiment analysis mechanism determines a sentiment associated with the medical event and drug name. The sentiment analysis mechanism generates medical clue metadata linking the sentiment with the medical event and the drug corresponding to the drug name and applies the medical clue metadata to analysis of other medical documents to identify sentiments associated with instances of the drug name or medical event in the other medical documents.

### BACKGROUND

The present application relates generally to an improved data processing apparatus and method and more specifically to mechanisms for performing sentiment analysis based on medical context.

Adverse drug reactions, or ADRs, are injuries caused to a patient because of the patient taking a drug (medication). An adverse event (AE), or adverse drug event (ADE), refers to any injury occurring at the time the patient is taking a medication, whether or not the medication itself is identified as the cause of the injury. Thus, an ADR is a special type of AE in which a causative relationship can be shown between the medication and the adverse reaction.

ADRs may occur following a single dose of the medication or due to a prolonged administration of a medication and may even be caused by the interaction of a combination of two or more medications that the patient may be taking. This is different from a “side effect” in that a “side effect” may comprise beneficial effects whereas ADRs are universally negative. The study of ADRs is the concern of the field known as pharmacovigilance.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising at least one processor and at least one memory, the at least one memory comprising instructions that are executed by the at least one processor to cause the at least one processor to be configured to implement a sentiment analysis mechanism for performing sentiment analysis of a medical event and a drug name within a medical document based on a medical context surrounding the medical event and the drug name. The method comprises analyzing a medical document to identify an occurrence of a medical event associated with a drug name. The method also comprises analyzing contextual content associated with the occurrence of the medical event and the drug name to identify one or more sentiment terms present in the contextual content. Moreover, the method comprises determining, based on a correlation of the one or more sentiment terms, the medical event, and the drug name, a sentiment associated with the medical event and drug name. The method also comprises generating medical clue metadata linking the sentiment with the medical event and the drug corresponding to the drug name. Additionally, the method comprises applying the medical clue metadata to analysis of other medical documents to identify sentiments associated with instances of the drug name or medical event in the other medical documents.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example block diagram illustrating components of a sentiment analysis mechanism in accordance with one illustrative embodiment;

FIG. 2 depicts a schematic diagram of one illustrative embodiment of a cognitive healthcare system in a computer network;

FIG. 3 is a block diagram of an example data processing system in which aspects of the illustrative embodiments are implemented; and

FIG. 4 is a flowchart outlining an example operation of a. sentiment analysis mechanism in accordance with one illustrative embodiment.

### DETAILED DESCRIPTION

Sentiment analysis has been used for personalized recommendations, client feedback tracking, brand analysis, precision marketing, and the like. The accuracy of sentiment analysis is crucial to the success of these applications. While sentiment analysis in the general domain has been extensively studied, little has been done today on how to perform sentiment analysis with high accuracy in the medical domain.

Sentiment analysis is generally known in the art. However, in the medical domain, typically human beings are required to manually go through spontaneous reports and identify adverse events. Further, sentiment classification in the medical domain uses sentiment for facilitating adverse event (AE) detection but does not make use of medical clues. That is, sentiment detection in known systems does not make use of medically relevant clues for the task of sentiment classification. Sentiments may be incorrectly identified especially in the medical context if one does not take into account medically relevant clues. For example, in the phrase “strong bitter taste” the term “strong” has a negative sentiment with the adverse event (AE) of “bitter taste”, yet the term “strong” has a positive sentiment in the phrase “strong pain killer” given the context of “pain killer”. Thus, indications of sentiment are different depending on the medical context. Moreover, known systems do not link sentiments to drugs and medical events.

Thus, the illustrative embodiments provide a sentiment analysis mechanism for the medical domain. The illustrative embodiments use medically relevant clues to detect sentiments and identify medical events correlated with a drug. A medical clue is a combination of a term with a medical event and a drug name. The illustrative embodiments are specifically directed to detection of sentiments with negative polarity in reports using medical clues obtained from the detection of medical events and corresponding annotations in the medical documentation. The illustrative embodiments link the sentiment to the medical event identified and to a drug being discussed to generate a medical clue. These medical clues may then be used as a basis for evaluating other documents as to their sentiment regarding drugs and medical events. Thus, the illustrative embodiments enable the discovery and use of medical clues to assist in sentiment analysis of medical documentation so as to properly evaluate sentiment to identify adverse events.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general-purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

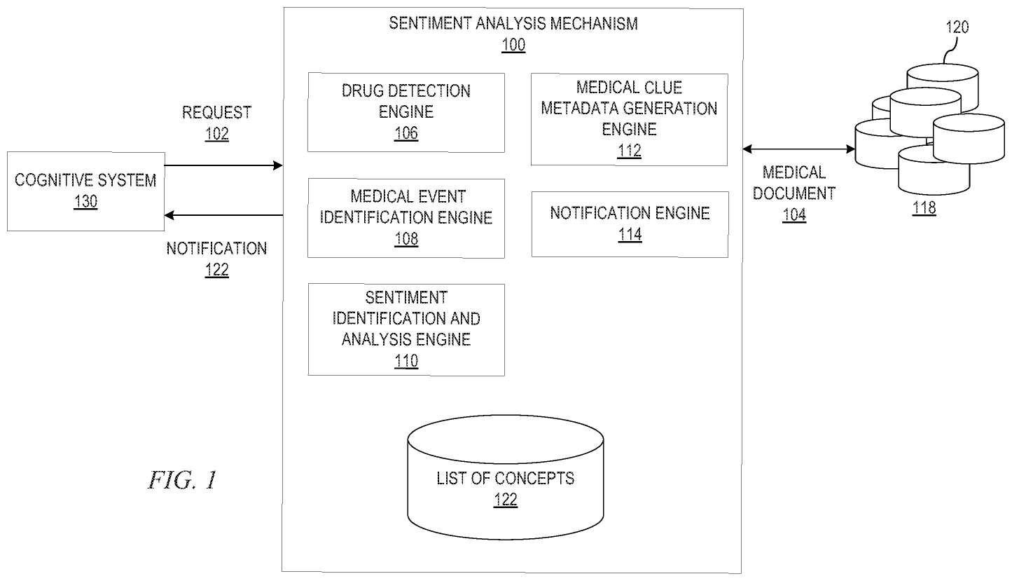

As noted above, the present invention provides mechanisms for performing sentiment analysis based on medical context. FIG. 1 is an example block diagram illustrating components of a sentiment analysis mechanism in accordance with one illustrative embodiment. As shown in FIG. 1, sentiment analysis mechanism 100, drug detection engine 106, medical event identification engine 108, sentiment identification and analysis engine 110, medical clue metadata generation engine 112, and notification engine 114.

Sentiment analysis mechanism 100 operates to automatically perform sentiment analysis of a medical event and a drug name within a medical document based on a medical context surrounding the medical event and the drug name. Thus, responsive to sentiment analysis mechanism 100 receiving a request 102 to perform sentiment analysis of medical document 104 from cognitive system 130, drug detection engine 106 and medical event identification engine 108 retrieve medical document 104 from corpora of data/information 118. Corpora of data/information 118 may be made up of one or more databases storing information about the electronic texts, documents, articles, websites, and the like. In once embodiment, corpora of data/information 118 may store medical documents such as patient electronic medical records or electronic health records. That is, these various sources themselves, different collections of sources, and the like, represent a different corpus 120 within the corpora 118. There may be different corpora 120 defined for different collections of documents based on various criteria depending upon the particular implementation. For example, different corpora may be established for different topics, subject matter categories, sources of information, or the like. As one example, a first corpus may be associated with healthcare documents while a second corpus may be associated with the Unified Medical Language System (UMLS) Metathesaurus. Alternatively, one corpus may be documents published by the U.S. Department of Health and Human Services while another corpus may be American Medical Association documents. Any collection of content having some similar attribute may be considered to be a corpus 120 within the corpora 118.

Once medical document 104 is retrieved, drug detection engine 106 detects one or more drugs names identified in list of concepts 122 that exist within medical document 104 utilizing a model, such as a Hierarchical Bayesian Model, to identify one or more topics that are directed to the one or more drug names. The Hierarchical Bayesian Model only considers, for the document under consideration, i.e. medical document 104, only the drugs and medical events mentioned in medical document 104. In order to identify one or more medical events associated with the one or more medications identified by drug detection engine 106, medical event identification engine 108 performs a similar operation but for one or more medical events from list of concepts 122. That is, utilizing Hierarchical Bayesian medical event identification engine 108 identifies one or more topics that are directed to the one or more medical events. Utilizing the same process as performed by drug detection engine 106, medical event identification engine 108 identifies how the one or more medical events are utilized in the topics of the one or more discussion forums as well as a medical event probability for each topic identified by the medication probability for each topic.

For each occurrence of a drug/medical event pair, sentiment identification and analysis engine 110 analyzes the context surrounding the occurrence of the medical event and the drug name to identify one or more sentiment terms present in the contextual content. That is, sentiment identification and analysis engine 110 analyzes the context surrounding the identified drug name and medical event for sentiment terms, which may also be referred to as medical clues thereby forming a medical clue. Thus, sentiment identification and analysis engine 110 links the sentiment to the identified medical event associated with the identified drug name being discussed to generate a medical clue, the medical clue is a combination of a term with a medical event and a drug name.

Based on the identified sentiment terms and medical clues, sentiment identification and analysis engine 110 generates a classification of the sentiment based on the word distributions for each sentiment (positive or negative) in medical document 104. It should be noted that a sentiment of medical document 104 as a whole may be used to evaluate the sentiment of the particular instance to determine a medical clue. Thus, sentiment identification and analysis engine 110 determines, based on a correlation of the one or more sentiment terms, the medical event, the drug name, and a sentiment (positive or negative) associated with the medical event/drug name thereby polarizing the associated medical clue.

Using the determined medical clue for each drug/medical event pair, medical clue metadata generation engine 114 generates medical clue metadata linking the sentiment with the medical event and the drug corresponding to the drug name. Medical clue metadata generation engine 114 stores the generated medical clue metadata data along with medical document 104 in corpora of data/information 118. By storing the medical clue metadata data along with medical document 104, then when medical document 104 is utilized in a cognitive operation by cognitive system 130, cognitive system 130 applies the medical clue metadata to analysis of other medical documents within corpora of data/information 118 to identify sentiments associated with instances of the drug name or medical event in the other medical documents. That is, cognitive system 130 utilizes the medical clue metadata to identify medical events, specified in the other medical documents, corresponding to the drug name and medical event.

Depending on the requested sentiment analysis of medical document 104, sentiment analysis mechanism 100 may also operate to generate and output a notification identifying one or more medical events/drug name pairs and their associated sentiment (positive or negative) identified within medical document 104. That is, based on the request, notification engine 114 generates an indication to one or more medical professionals of one or more medical events/drug name pairs and their associated sentiment (positive or negative) identified within the identified medical document 104, so that, especially if the sentiment is one of a negative nature thereby indicating an adverse event, the medical professionals associated with the drug under consideration may address the identified adverse event associated with the drug.

Thus, the medical clues may then be used as a basis for identify other pairings of medical events with drugs and used to evaluate sentiment in not only the identified document, but other documents, social networking website content, patient forums, or the like. In this way, instances in documents of medical events with drugs that have a negative sentiment may be flagged as potential adverse events. These adverse events may be reported to appropriate personnel, e.g., doctors, pharmaceutical companies, or the like. In some cases, drug manufactures may be informed of adverse events that they may previously not have been aware of.

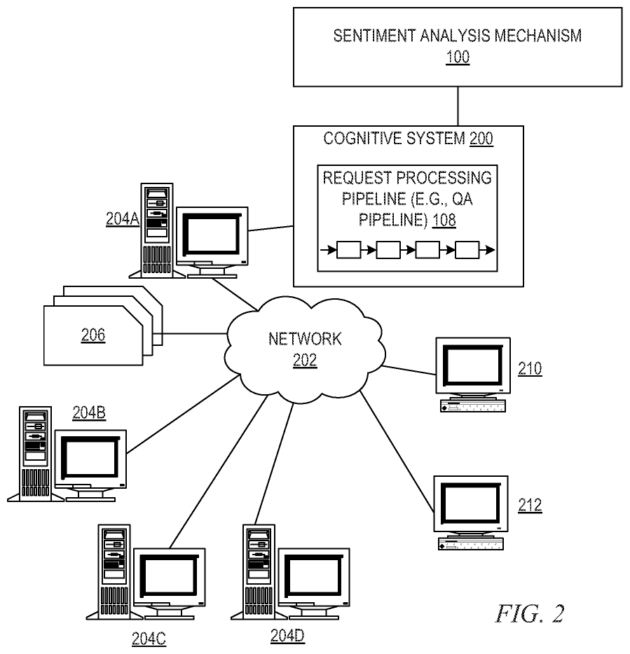

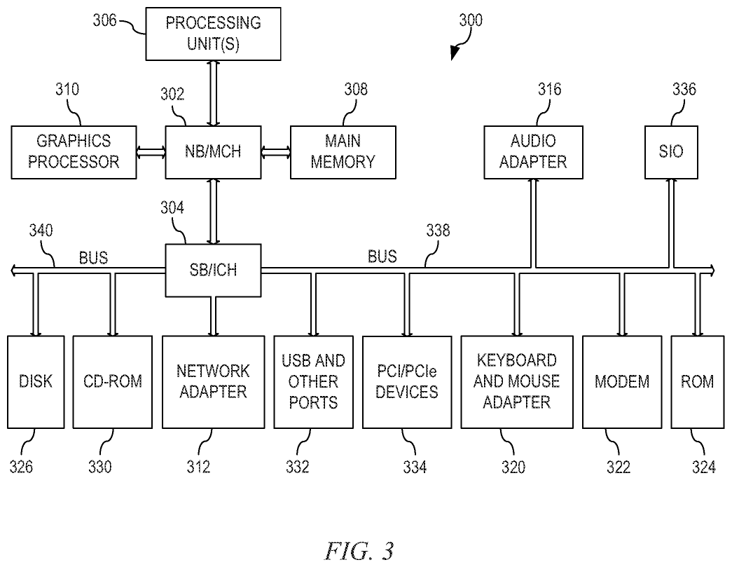

It is clear from the above, that the illustrative embodiments may be utilized in many different types of data processing environments. In order to provide a context for the description of the specific elements and functionality of the illustrative embodiments, FIGS. 2-3 are provided hereafter as example environments in which aspects of the illustrative embodiments may be implemented. It should be appreciated that FIGS. 2-3 are only examples and are not intended to assert or imply any limitation with regard to the environments in which aspects or embodiments of the present invention may be implemented. Many modifications to the depicted environments may be made without departing from the spirit and scope of the present invention.

It should be noted that the mechanisms of the illustrative embodiments need not be utilized with a cognitive system. To the contrary, the illustrative embodiments may be implemented as a standalone sentiment analysis mechanism implemented on one or more computing devices or systems. The standalone sentiment analysis mechanism may generate an output notification that may be utilized by a user when evaluating a particular drug, adverse event, or the combination of drug and adverse event. Thus, in a standalone implementation, the sentiment analysis mechanism may be implemented using one or more computing devices or systems such as depicted in FIG. 3, as one example. However, to illustrate further functionality of illustrative embodiments of the present invention, FIGS. 2-3 are provided to illustrate the way in which the sentiment analysis mechanism may be utilized with a cognitive system to perform cognitive healthcare operations for performing sentiment analysis of a medical event and a drug name within a medical document based on a medical context surrounding the medical event and the drug name.

FIGS. 2-3 are directed to describing an example cognitive system for healthcare applications (also referred to herein as a “healthcare cognitive system”) which implements a request processing pipeline, such as a Question Answering (QA) pipeline (also referred to as a Question/Answer pipeline or Question and Answer pipeline) for example, request processing methodology, and request processing computer program product with which the mechanisms of the illustrative embodiments are implemented. These requests may be provided as structured or unstructured request messages, natural language questions, or any other suitable format for requesting an operation to be performed by the healthcare cognitive system. As described in more detail hereafter, the particular healthcare application that is implemented in the cognitive system of the present invention is a healthcare application for performing sentiment analysis of a medical event and a drug name within a medical document based on a medical context surrounding the medical event and the drug name by the sentiment analysis mechanism of the illustrative embodiments.

It should be appreciated that the healthcare cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests (or questions in implementations using a QA pipeline), depending on the desired implementation. For example, in some cases, a first request processing pipeline may be trained to operate on input requests directed to a first medical malady domain (e.g., medication interactions) while another request processing pipeline may be trained to answer input requests in another medical malady domain (e.g., seriousness associated with medications). In other cases, for example, the request processing pipelines may be configured to provide different types of cognitive functions or support different types of healthcare applications, such as one request processing pipeline being used for adverse events, another request processing pipeline being configured for seriousness, another request processing pipeline being configured for expectedness, etc.

Moreover, each request processing pipeline may have their own associated corpus or corpora that they ingest and operate on, e.g., one corpus for adverse event documents, another corpus for seriousness related documents, and another for expectedness documents in the above examples. In some cases, the request processing pipelines may each operate on the same domain of input questions but may have different configurations, e.g., different annotators or differently trained annotators, such that different analysis and potential answers are generated. The healthcare cognitive system may provide additional logic for routing input questions to the appropriate request processing pipeline, such as based on a determined domain of the input request, combining and evaluating final results generated by the processing performed by multiple request processing pipelines, and other control and interaction logic that facilitates the utilization of multiple request processing pipelines.

The request processing pipelines may utilize the analysis performed by the sentiment analysis mechanism of one or more of the illustrative embodiments, such as sentiment analysis mechanism 100 in FIG. 1, as a factor considered by the request processing pipeline when performing cognitive evaluations of a patient to automatically perform sentiment analysis of a medical event and a drug name within a medical document based on a medical context surrounding the medical event and the drug name, with an aim at minimizing adverse drug reactions for drugs taken by the patient.

As noted above, one type of request processing pipeline with which the mechanisms of the illustrative embodiments may be utilized is a Question Answering (QA) pipeline. The description of example embodiments of the present invention hereafter will utilize a QA pipeline as an example of a request processing pipeline that may be augmented to include mechanisms in accordance with one or more illustrative embodiments for performing sentiment analysis of a medical event and a drug name within a medical document based on a medical context surrounding the medical event and the drug name by the sentiment analysis mechanism of the illustrative embodiments. It should be appreciated that while embodiments of the present invention will be described in the context of the cognitive system implementing one or more QA pipelines that operate on an input question, the illustrative embodiments are not limited to such. Rather, the mechanisms of the illustrative embodiments may operate on requests that are not posed as “questions” but are formatted as requests for the cognitive system to perform cognitive operations on a specified set of input data using the associated corpus or corpora and the specific configuration information used to configure the cognitive system. For example, rather than asking a natural language question of “What diagnosis applies to patient P?”, the cognitive system may instead receive a request of “generate diagnosis for patient P,” or the like. It should be appreciated that the mechanisms of the QA system pipeline may operate on requests in a similar manner to that of input natural language questions with minor modifications. In fact, in some cases, a request may be converted to a natural language question for processing by the QA system pipelines if desired for the particular implementation.

Thus, it is important to first have an understanding of how cognitive systems and question and answer creation in a cognitive system implementing a QA pipeline is implemented before describing how the mechanisms of the illustrative embodiments are integrated in and augment such cognitive systems and request processing pipeline, or QA pipeline, mechanisms. It should be appreciated that the mechanisms described in FIGS. 2-3 are only examples and are not intended to state or imply any limitation with regard to the type of cognitive system mechanisms with which the illustrative embodiments are implemented. Many modifications to the example cognitive system shown in FIGS. 2-3 may be implemented in various embodiments of the present invention without departing from the spirit and scope of the present invention.

As an overview, a cognitive system is a specialized computer system, or set of computer systems, configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation, e.g., items of interest to a particular user, potential new contact recommendations, or the like.

IBM Watson™ is an example of one such cognitive system which can process human readable language and identify inferences between text passages with human-like high accuracy at speeds far faster than human beings and on a larger scale. In general, such cognitive systems are able to perform the following functions:
Navigate the complexities of human language and understanding,
Ingest and process vast amounts of structured and unstructured data,
Generate and evaluate hypothesis,
Weigh and evaluate responses that are based only on relevant evidence,
Provide situation-specific advice, insights, and guidance,
Improve knowledge and learn with each iteration and interaction through machine learning processes,
Enable decision making at the point of impact (contextual guidance),
Scale in proportion to the task,
Extend and magnify human expertise and cognition,
Identify resonating, human-like attributes and traits from natural language,
Deduce various language specific or agnostic attributes from natural language,
High degree of relevant recollection from data points (images, text, voice) (memorization and recall),
Predict and sense with situational awareness that mimic human cognition based on experiences, or
Answer questions based on natural language and specific evidence.

In one aspect, cognitive systems provide mechanisms for answering questions posed to these cognitive systems using a Question Answering pipeline or system (QA system) and/or process requests which may or may not be posed as natural language questions. The QA pipeline or system is an artificial intelligence application executing on data processing hardware that answers questions pertaining to a given subject-matter domain presented in natural language. The QA pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the QA pipeline. The document may include any file, text, article, or source of data for use in the QA system. For example, a QA pipeline accesses a body of knowledge about the domain, or subject matter area, e,g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users input questions to cognitive system which implements the QA pipeline. The QA pipeline then answers the input questions using the content in the corpus of data by evaluating documents, sections of documents, portions of data in the corpus, or the like, When a process evaluates a given section of a document for semantic content, the process can use a variety of conventions to query such document from the QA pipeline, e.g., sending the query to the QA pipeline as a well-formed question which is then interpreted by the QA pipeline and a response is provided containing one or more answers to the question. Semantic content is content based on the relation between signifiers, such as words, phrases, signs, and symbols, and what they stand for, their denotation, or connotation. In other words, semantic content is content that interprets an expression, such as by using Natural Language Processing.

As will be described in greater detail hereafter, the QA pipeline receives an input question, parses the question to extract the major features of the question, uses the extracted features to formulate queries, and then applies those queries to the corpus of data. Based on the application of the queries to the corpus of data, the QA pipeline generates a set of hypotheses, or candidate answers to the input question, by looking across the corpus of data for portions of the corpus of data that have some potential for containing a valuable response to the input question. The QA pipeline then performs deep analysis on the language of the input question and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the input question and the found portions of the corpus of data. Other reasoning algorithms may look at temporal or spatial features in the language, while others may evaluate the source of the portion of the corpus of data and evaluate its veracity.

The scores obtained from the various reasoning algorithms indicate the extent to which the potential response is inferred by the input question based on the specific area of focus of that reasoning algorithm. Each resulting score is then weighted against a statistical model. The statistical model captures how well the reasoning algorithm performed at establishing the inference between two similar passages for a particular domain during the training period of the QA pipeline. The statistical model is used to summarize a level of confidence that the QA pipeline has regarding the evidence that the potential response, i.e. candidate answer, is inferred by the question. This process is repeated for each of the candidate answers until the QA pipeline identifies candidate answers that surface as being significantly stronger than others and thus, generates a final answer, or ranked set of answers, for the input question.

As mentioned above, QA pipeline mechanisms operate by accessing information from a corpus of data or information (also referred to as a corpus of content), analyzing it, and then generating answer results based on the analysis of this data. Accessing information from a corpus of data typically includes: a database query that answers questions about what is in a collection of structured records, and a search that delivers a collection of document links in response to a query against a collection of unstructured data (text, markup language, etc.). Conventional question answering systems are capable of generating answers based on the corpus of data and the input question, verifying answers to a collection of questions for the corpus of data, correcting errors in digital text using a corpus of data, and selecting answers to questions from a pool of potential answers, i.e. candidate answers.

Content creators, such as article authors, electronic document creators, web page authors, document database creators, and the like, determine use cases for products, solutions, and services described in such content before writing their content. Consequently, the content creators know what questions the content is intended to answer in a particular topic addressed by the content. Categorizing the questions, such as in terms of roles, type of information, tasks, or the like, associated with the question, in each document of a corpus of data allows the QA pipeline to more quickly and efficiently identify documents containing content related to a specific query. The content may also answer other questions that the content creator did not contemplate that may be useful to content users. The questions and answers may be verified by the content creator to be contained in the content for a given document. These capabilities contribute to improved accuracy, system performance, machine learning, and confidence of the QA pipeline. Content creators, automated tools, or the like, annotate or otherwise generate metadata for providing information useable by the QA pipeline to identify these questions and answer attributes of the content.

Operating on such content, the QA pipeline generates answers for input questions using a plurality of intensive analysis mechanisms which evaluate the content to identify the most probable answers, i.e. candidate answers, for the input question. The most probable answers are output as a ranked listing of candidate answers ranked according to their relative scores or confidence measures calculated during evaluation of the candidate answers, as a single final answer having a highest-ranking score or confidence measure, or which is a best match to the input question, or a combination of ranked listing and final answer.

With regard to the sentiment analysis mechanism of the illustrative embodiments, the information generated by the sentiment analysis mechanism may be input to the QA pipeline for use as yet another portion of the corpus or corpora upon which the QA pipeline operates. For example, the information generated by the sentiment analysis mechanism may be included in inputs upon which the operations of the reasoning algorithms are applied, as part of the evaluation of evidence supporting various candidate answers or responses generated by the QA pipeline, or the like. Thus, the reasoning algorithms may include factors for performing sentiment analysis of a medical event and a drug name within a medical document based on a medical context surrounding the medical event and the drug name.

FIG. 2 depicts a schematic diagram of one illustrative embodiment of a cognitive system 200 implementing a request processing pipeline 208, which in some embodiments may be a question answering (QA) pipeline, in a computer network 202. For purposes of the present description, it will be assumed that the request processing pipeline 208 is implemented as a QA pipeline that operates on structured and/or unstructured requests in the form of input questions. One example of a question processing operation which may be used in conjunction with the principles described herein is described in U.S. Patent Application Publication No. 2011/0125734, which is herein incorporated by reference in its entirety. The cognitive system 200 is implemented on one or more computing devices 204A-D (comprising one or more processors and one or more memories, and potentially any other computing device elements generally known in the art including buses, storage devices, communication interfaces, and the like) connected to the computer network 202. For purposes of illustration only, FIG. 2 depicts the cognitive system 200 being implemented on computing device 204A only, but as noted above the cognitive system 200 may be distributed across multiple computing devices, such as a plurality of computing devices 204A-D. The network 202 includes multiple computing devices 204A-D, which may operate as server computing devices, and 210-212 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 200 and network 202 enables question processing and answer generation (QA) functionality for one or more cognitive system users via their respective computing devices 210-212. In other embodiments, the cognitive system 200 and network 202 may provide other types of cognitive operations including, but not limited to, request processing and cognitive response generation which may take many different forms depending upon the desired implementation, e.g., cognitive information retrieval, training/instruction of users, cognitive evaluation of data, or the like, Other embodiments of the cognitive system 200 may be used with components, systems, sub-systems, and/or devices other than those that are depicted herein.

The cognitive system 200 is configured to implement a request processing pipeline 208 that receive inputs from various sources. The requests may be posed in the form of a natural language question, natural language request for information, natural language request for the performance of a cognitive operation, or the like. For example, the cognitive system 200 receives input from the network 202, a corpus or corpora of electronic documents 206, cognitive system users, and/or other data and other possible sources of input. In one embodiment, some or all of the inputs to the cognitive system 200 are routed through the network 202. The various computing devices 204A-D on the network 202 include access points for content creators and cognitive system users. Some of the computing devices 204A-D include devices for a database storing the corpus or corpora of data 206 (which is shown as a separate entity in FIG. 2 for illustrative purposes only). Portions of the corpus or corpora of data 206 may also be provided on one or more other network attached storage devices, in one or more databases, or other computing devices not explicitly shown in FIG. 2. The network 202 includes local network connections and remote connections in various embodiments, such that the cognitive system 200 may operate in environments of any size, including local and global, e.g., the Internet.

In one embodiment, the content creator creates content in a document of the corpus or corpora of data 206 for use as part of a corpus of data with the cognitive system 200. The document includes any file, text, article, or source of data for use in the cognitive system 200. Cognitive system users access the cognitive system 200 via a network connection or an Internet connection to the network 202, and input questions/requests to the cognitive system 200 that are answered/processed based on the content in the corpus or corpora of data 206. In one embodiment, the questions/requests are formed using natural language. The cognitive system 200 parses and interprets the question/request via a pipeline 208, and provides a response to the cognitive system user, e.g., cognitive system user 210, containing one or more answers to the question posed, response to the request, results of processing the request, or the like. In some embodiments, the cognitive system 200 provides a response to users in a ranked list of candidate answers/responses while in other illustrative embodiments, the cognitive system 200 provides a single final answer/response or a combination of a final answer/response and ranked listing of other candidate answers/responses.

The cognitive system 200 implements the pipeline 208 which comprises a plurality of stages for processing an input question/request based on information obtained from the corpus or corpora of data 206. The pipeline 208 generates answers/responses for the input question or request based on the processing of the input question/request and the corpus or corpora of data 206.

In some illustrative embodiments, the cognitive system 200 may be the IBM Watson™ cognitive system available from International Business Machines Corporation of Armonk, N.Y., which is augmented with the mechanisms of the illustrative embodiments described hereafter. As outlined previously, a pipeline of the IBM Watson™ cognitive system receives an input question or request which it then parses to extract the major features of the question/request, which in turn are then used to formulate queries that are applied to the corpus or corpora of data 206. Based on the application of the queries to the corpus or corpora of data 206, a set of hypotheses, or candidate answers/responses to the input question/request, are generated by looking across the corpus or corpora of data 206 for portions of the corpus or corpora of data 206 (hereafter referred to simply as the corpus 206) that have some potential for containing a valuable response to the input question/response (hereafter assumed to be an input question). The pipeline 208 of the IBM Watson™ cognitive system then performs deep analysis on the language of the input question and the language used in each of the portions of the corpus 206 found during the application of the queries using a variety of reasoning algorithms.

The scores obtained from the various reasoning algorithms are then weighted against a statistical model that summarizes a level of confidence that the pipeline 208 of the IBM Watson™ cognitive system 200, in this example, has regarding the evidence that the potential candidate answer is inferred by the question. This process is repeated for each of the candidate answers to generate a ranked listing of candidate answers which may then be presented to the user that submitted the input question, e.g., a user of client computing device 210, or from which a final answer is selected and presented to the user. More information about the pipeline 208 of the IBM Watson™ cognitive system 200 may be obtained, for example, from the IBM Corporation website, IBM Redbooks, and the like. For example, information about the pipeline of the IBM Watson™ cognitive system can be found in Yuan et al., “Watson and Healthcare,” IBM developerWorks, 2011 and “The Era of Cognitive Systems: An Inside Look at IBM Watson and How it Works” by Rob High, IBM Redbooks, 2012.

As noted above, while the input to the cognitive system 200 from a client device may be posed in the form of a natural language question, the illustrative embodiments are not limited to such. Rather, the input question may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result. In particular, the mechanisms of the healthcare based cognitive system may process medication-adverse events or medication-adverse drug reaction pairings when performing the healthcare oriented cognitive system result, e.g., a diagnosis or treatment recommendation.

In the context of the present invention, cognitive system 200 may provide a cognitive functionality for performing sentiment analysis of a medical event and a drug name within a medical document based on a medical context surrounding the medical event and the drug name. Thus, the cognitive system 200 may be a healthcare cognitive system 200 that operates in the medical or healthcare type domains and which may process requests for such healthcare operations via the request processing pipeline 208 input as either structured or unstructured requests, natural language input questions, or the like. In one illustrative embodiment, the cognitive system 200 is a medication analysis system that analyzes medical documents to identify discussion medical events related to a drug under consideration, and further analyze natural language text within the discussion forums in order to automatically perform sentiment analysis of a medical event and a drug name within a medical document based on a medical context surrounding the medical event and the drug name.

As shown in FIG. 2, the cognitive system 200 is further augmented, in accordance with the mechanisms of the illustrative embodiments, to include logic implemented in specialized hardware, software executed on hardware, or any combination of specialized hardware and software executed on hardware, for implementing sentiment analysis mechanism 100. As described previously, sentiment analysis mechanism 100 provides a probabilistic model to analyze medical documents for concepts, where the probabilistic model combines, for each word, seriousness, adverse drug reaction, and medication expectedness probabilistic models, replacing individual models with one combined model that generates an indication of a probability that the content of the medical document indicates an actual adverse event. Sentiment analysis mechanism 100 identifies a difference between adverse events in the medical documents based on sentiment of the surrounding context. The illustrative embodiments automatically identify, via medical documents, adverse events potentially caused by a medication, which the medication manufacturer may not previously know about.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 3 is provided as an example of one type of data processing system in which aspects of the present invention may be implemented. Many other types of data processing systems may be likewise configured to specifically implement the mechanisms of the illustrative embodiments.

FIG. 3 is a block diagram of an example data processing system in which aspects of the illustrative embodiments are implemented. Data processing system 300 is an example of a computer, such as server 204A or client 210 in FIG. 2, in which computer usable code or instructions implementing the processes for illustrative embodiments of the present invention are located. In one illustrative embodiment, FIG. 3 represents a server computing device, such as a server 204, which, which implements a cognitive system 200 and QA system pipeline 208 augmented to include the additional mechanisms of the illustrative embodiments described hereafter.

In the depicted example, data processing system 300 employs a hub architecture including North Bridge and Memory Controller Hub (NB/MCH) 302 and South Bridge and Input/Output (I/O) Controller Hub (SB/ICH) 304, Processing unit 306, main memory 308, and graphics processor 310 are connected to NB/MCH 302, Graphics processor 310 is connected to NB/MCH 302 through an accelerated graphics port (ACIP).

In the depicted example, local area network (LAN) adapter 312 connects to SB/ICH 304. Audio adapter 316, keyboard and mouse adapter 320, modem 322, read only memory (ROM) 324, hard disk drive (HDD) 326, CD-ROM drive 330, universal serial bus (USB) ports and other communication ports 332, and PCI/PCIe devices 334 connect to SB/ICH 304 through bus 338 and bus 340. PCI/PCIe devices may include, for example, Ethernet adapters, add-in cards, and PC cards for notebook computers. PCI uses a card bus controller, while PCIe does not. ROM 324 may be, for example, a flash basic input/output system (BIOS).

HDD 326 and CD-ROM drive 330 connect to SB/ICH 304 through bus 340. HDD 326 and CD-ROM drive 330 may use, for example, an integrated drive electronics (IDE) or serial advanced technology attachment (SATA) interface. Super I/O (SIO) device 336 is connected to SB/ICH 304.

An operating system runs on processing unit 306. The operating system coordinates and provides control of various components within the data processing system 300 in FIG. 3. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 300.

As a server, data processing system 300 may be, for example, an IBM® eServer™ System p® computer system, running the Advanced Interactive Executive (AIX®) operating system or the LINUX® operating system. Data processing system 300 may be a symmetric multiprocessor (SMP) system including a plurality of processors in processing unit 306. Alternatively, a single processor system may be employed.

Instructions for the operating system, the object-oriented programming system, and applications or programs are located on storage devices, such as HDD 326, and are loaded into main memory 308 for execution by processing unit 306. The processes for illustrative embodiments of the present invention are performed by processing unit 306 using computer usable program code, which is located in a memory such as, for example, main memory 308, ROM 324, or in one or more peripheral devices 326 and 330, for example.

A bus system, such as bus 338 or bus 340 as shown in FIG. 3, is comprised of one or more buses. Of course, the bus system may be implemented using any type of communication fabric or architecture that provides for a transfer of data between different components or devices attached to the fabric or architecture. A communication unit, such as modem 322 or network adapter 312 of FIG. 3, includes one or more devices used to transmit and receive data. A memory may be, for example, main memory 308, ROM 324, or a cache such as found in NB/MCH 302 in FIG. 3.

Those of ordinary skill in the art will appreciate that the hardware depicted in FIGS. 2 and 3 may vary depending on the implementation. Other internal hardware or peripheral devices, such as flash memory, equivalent non-volatile memory, or optical disk drives and the like, may be used in addition to or in place of the hardware depicted in FIGS. 2 and 3. Also, the processes of the illustrative embodiments may be applied to a multiprocessor data processing system, other than the SMP system mentioned previously, without departing from the spirit and scope of the present invention.

Moreover, the data processing system 300 may take the form of any of a number of different data processing systems including client computing devices, server computing devices, a tablet computer, laptop computer, telephone or other communication device, a personal digital assistant (PDA), or the like. In some illustrative examples, data processing system 300 may be a portable computing device that is configured with flash memory to provide non-volatile memory for storing operating system files and/or user-generated data, for example. Essentially, data processing system 300 may be any known or later developed data processing system without architectural limitation.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

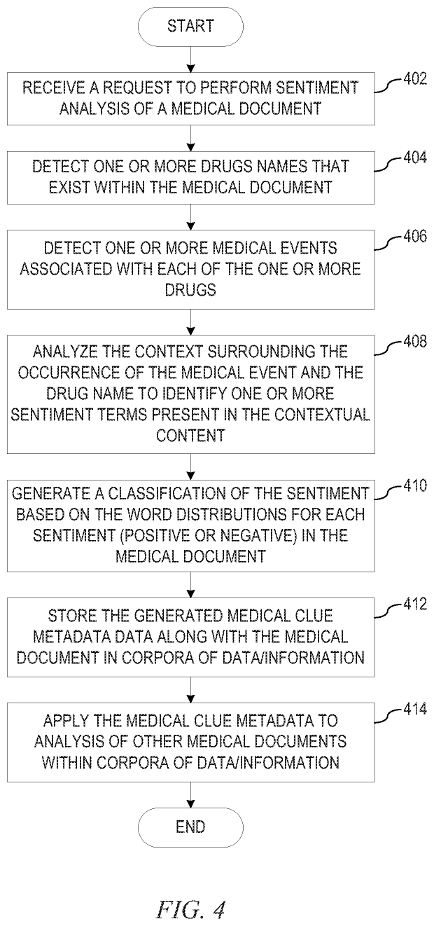

FIG. 4 is a flowchart outlining an example operation of a sentiment analysis mechanism in accordance with one illustrative embodiment. As the exemplary operation begins, the sentiment analysis mechanism receives a request to perform sentiment analysis of a medical document (step 402). The sentiment analysis mechanism detects one or more drugs names that exist within the medical document (step 404) as well as detect one or more medical events associated with each of the one or more drugs (step 406). For each occurrence of a drug/medical event pair, the sentiment analysis mechanism analyzes the context surrounding the occurrence of the medical event and the drug name to identify one or more sentiment terms present in the contextual content (step 408). That is, the sentiment analysis mechanism analyzes the context surrounding the identified drug name and medical event to determined one or more sentiment terms, thereby forming a medical clue. Thus, the sentiment analysis mechanism links the sentiment to the identified medical event associated with the identified drug name being discussed to generate a medical clue, the medical clue is a combination of a term with a medical event and a drug name.

Based on the identified sentiment terms and medical clues, the sentiment analysis mechanism generates a classification of the sentiment based on the word distributions for each sentiment (positive or negative) in the medical document (step 410). The sentiment analysis mechanism stores the generated medical clue metadata data along with the medical document in corpora of data/information (step 412). By storing the medical clue metadata data along with the medical document, then, when the medical document is utilized in a cognitive operation by a cognitive system, the cognitive system applies the medical clue metadata to analysis of other medical documents within corpora of data/information (step 414) to identify sentiments associated with instances of the drug name or medical event in the other medical documents. That is, the cognitive system utilizes the medical clue metadata to identify medical events, specified in the other medical documents, corresponding to the drug name and medical event. The operation ends thereafter.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Moderns, cable moderns and Ethernet cards are just a few of the currently available types of network adapters for wired communications, Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and at least one memory, the at least one memory comprising instructions that are executed by the at least one processor to cause the at least one processor to be configured to implement a sentiment analysis mechanism for performing sentiment analysis of a medical event and a drug name within a medical document based on a medical context surrounding the medical event and the drug name, the method comprising:
analyzing a medical document to identify an occurrence of a medical event associated with a drug name;
analyzing contextual content associated with the occurrence of the medical event and the drug name to identify one or more sentiment terms present in the contextual content;
determining, based on a correlation of the one or more sentiment terms, the medical event, and the drug name, a sentiment associated with the medical event and drug name;
generating medical clue metadata linking the sentiment with the medical event and the drug corresponding to the drug name; and
applying the medical clue metadata to analysis of other medical documents to identify sentiments associated with instances of the drug name or medical event in the other medical documents.

2. The method of claim 1, wherein determining the sentiment comprises:
classifying the sentiment terms into positive and negative sentiment terms; and
determining the sentiment of the occurrence of the medical event and the drug name based on the classification of the sentiment terms.

3. The method of claim 1, wherein determining the sentiment comprises:
classifying a sentiment of the document as a whole; and
determining the sentiment of the occurrence of the medical event and the drug name based on the classification of the sentiment of the document as a whole.

4. The method of claim 1, wherein applying the medical clue metadata to analysis of other medical documents comprises identifying a medical events specified in the other medical documents, corresponding to the drug name and the medical event.

5. The method of claim 1, further comprising:
responsive to the sentiment associated with a particular medical event and drug name being negative, outputting a notification identifying the medical even as an adverse event.

6. The method of claim 1, wherein the medical clue metadata linking the sentiment with the medical event and the drug corresponding to the drug name are stored with the medical document.

7. The method of claim 1, wherein the other medical documents comprise patient medical records.

8. A computer program product comprising a computer readable storage medium having a computer readable program stored therein, wherein the computer readable program, when executed on a data processing system, causes the data processing system to implement a sentiment analysis mechanism for performing sentiment analysis of a medical event and a drug name within a medical document based on a medical context surrounding the medical event and the drug name, and further causes the data processing system to:
analyze a medical document to identify an occurrence of a medical event associated with a drug name;
analyze contextual content associated with the occurrence of the medical event and the drug name to identify one or more sentiment terms present in the contextual content;
determine, based on a correlation of the one or more sentiment terms, the medical event, and the drug name, a sentiment associated with the medical event and drug name;
generate medical clue metadata linking the sentiment with the medical event and the drug corresponding to the drug name; and
apply the medical clue metadata to analysis of other medical documents to identify sentiments associated with instances of the drug name or medical event in the other medical documents.

9. The computer program product of claim 8, wherein the computer readable program to determine the sentiment further causes the data processing system to:
classify the sentiment terms into positive and negative sentiment terms; and
determine the sentiment of the occurrence of the medical event and the drug name based on the classification of the sentiment terms.

10. The computer program product of claim 8, wherein the computer readable program to determine the sentiment further causes the data processing system to:
classify a sentiment of the document as a whole; and
determine the sentiment of the occurrence of the medical event and the drug name based on the classification of the sentiment of the document as a whole.

11. The computer program product of claim 8, wherein the computer readable program to apply the medical clue metadata to analysis of other medical documents further causes the data processing system to identify a medical events specified in the other medical documents, corresponding to the drug name and the medical event.

12. The computer program product of claim 8, wherein the computer readable program further causes the data processing system to:
responsive to the sentiment associated with a particular medical event and drug name being negative, output a notification identifying the medical even as an adverse event.

13. The computer program product of claim 8, wherein the medical clue metadata linking the sentiment with the medical event and the drug corresponding to the drug name are stored with the medical document.

14. The computer program product of claim 8, wherein the other medical documents comprise patient medical records.

15. A data processing system comprising:
at least one processor; and
at least one memory coupled to the at least one processor, wherein the at least one memory comprises instructions which, when executed by the at least one processor, cause the at least one processor to implement a sentiment analysis mechanism for performing sentiment analysis of a medical event and a drug name within a medical document based on a medical context surrounding the medical event and the drug name, and further cause the at least one processor to:
analyze a medical document to identify an occurrence of a medical event associated with a drug name;
analyze contextual content associated with the occurrence of the medical event and the drug name to identify one or more sentiment terms present in the contextual content;
determine, based on a correlation of the one or more sentiment terms, the medical event, and the drug name, a sentiment associated with the medical event and drug name;
generate medical clue metadata linking the sentiment with the medical event and the drug corresponding to the drug name; and
apply the medical clue metadata to analysis of other medical documents to identify sentiments associated with instances of the drug name or medical event in the other medical documents.

16. The data processing system of claim 15, wherein the instructions to determine the sentiment further cause the at least one processor to:
classify the sentiment. terms into positive and negative sentiment terms; and
determine the sentiment of the occurrence of the medical event and the drug name based on the classification of the sentiment terms.

17. The data processing system of claim 15, wherein the instructions to determine the sentiment further cause the at least one processor to:
classify a sentiment of the document as a whole; and
determine the sentiment of the occurrence of the medical event and the drug name based on the classification of the sentiment of the document as a whole.

18. The data processing system of claim 15, wherein the instructions to apply the medical clue metadata to analysis of other medical documents further cause the at least one processor to identify a medical events specified in the other medical documents, corresponding to the drug name and the medical event.

19. The data processing system of claim 15, wherein the instructions further causes the at least one processor to:
responsive to the sentiment associated with a particular medical event and drug name being negative, output a notification identifying the medical even as an adverse event.

20. The data processing system of claim 15, wherein the medical clue metadata linking the sentiment with the medical event and the drug corresponding to the drug name are stored with the medical document.
